(19) United States
(12) Patent Application Publication
(10) Pub. No.: US 2001/0051317 A1
(43) Pub. Date: Dec. 13, 2001

(54) Charged-particle-beam optical systems including beam tube exhibiting reduced eddy currents

(75) Inventor: Katsushi Nakano, Kawasaki-shi (JP)

Correspondence Address: KLARQUIST SPARKMAN CAMPBELL, LEIGH & WHINSTON, LLP, One World Trade center, 121 S.W. Salmon Street, Suite 1600, Portland, OR, 97204-2988, US

(73) Assignee: Nikon Corporation

(21) Appl. No.: 09/875,330

(22) Filed: Jun. 5, 2001

(30) Foreign Application Priority Data
Jun. 5, 2000 (JP) 2000-167436

(51) Int. Cl. G03C 5/00; A61N 5/00; G21G 5/00

(52) U.S. Cl. 430/296; 430/942; 250/492.2

(57) ABSTRACT

Charged-particle-beam (CPB) optical systems are disclosed that exhibit reduced eddy currents forming in the beam tube of the system. The eddy currents otherwise would degrade beam-control response time of the system. In an embodiment, the beam tube defines at least one slit in an “eddy-current zone” of the beam tube adjacent an energizable coil of the system, such as a deflector coil. The slit(s) is situated so as to divide the eddy-current zone. The slit(s) extends at least part way through the thickness dimension of the beam tube and can be formed using conventional machine tools, wire cutting, or electrical-discharge machining, or other suitable technique. Compared to an eddy-current zone lacking a slit, the divided eddy-current zones produced by the slit(s) have substantially reduced overall area, thereby reducing eddy current in the beam tube and allowing a corresponding increase in beam-control speed.

### FIELD OF THE INVENTION

This invention pertains to methods and apparatus for transferring a pattern to a sensitive substrate using a charged particle beam. This technology, generally termed “microlithography,” is a key technology utilized in the manufacture of microelectronic devices such as integrated circuits, displays, thin-film magnetic pickup heads, and micromachines, for example.

The “substrate” can be any of various materials such as a silicon or gallium arsenide wafer, glass, or the like. By “sensitive” is meant that the substrate includes a layer of a material (termed a “resist”) that responds to exposure by the charged particle beam in a way that results in imprinting of an image or of a pattern in the resist. The charged particle beam can be an electron beam or ion beam.

### BACKGROUND OF THE INVENTION

In recent years, with the increased miniaturization and density of microelectronic devices, an urgent need has arisen for microlithographic apparatus and methods capable of transferring a pattern to a sensitive substrate with an accuracy and resolution greater than achievable using light (including ultraviolet light). The main reasons for considering use of a charged particle beam for pattern transfer are similar to the reasons for which electron microscopy achieves better image resolution than light microscopy.

In charged-particle-beam (CPB) microlithography, the charged particle beam can be used for pattern drawing, in which the pattern is inscribed line-by-line on the substrate (this technique has low throughput and hence now is used mostly for making reticles and masks), or for pattern projection-exposure (in which a pattern, defined by a reticle or mask, is projection-exposed onto the substrate). Because a charged particle beam has a shorter wavelength than light, the charged particle beam potentially can be used for lithographically forming a pattern comprising shapes and forms that are much smaller than possible using light.

A charged particle beam cannot be transmitted through the atmosphere. As a result, microlithography performed using a charged particle beam must be performed inside a vacuum chamber. The walls of the vacuum chamber typically are configured as a “liner tube” extending along an optical axis and defining a space that is evacuated to a suitably high vacuum. The charged particle beam propagates generally in an axial direction, within the lumen of the liner tube.

Whereas certain CPB optical components (such as aperture diaphragms) are located within the liner tube, many components, including CPB lenses and CPB deflectors, are located outside the liner tube. CPB lenses and deflectors serve to converge and deflect the charged particle beam as required within the liner tube. Each CPB lens or deflector typically comprises one or more electrically energizable coils. When energized, the coil generates a magnetic field. Certain coils can be used as deflectors, for example, for deflecting the charged particle beam as required for scanning exposures and pattern “writing.” Other coils, incorporated into a dynamic-focus system, serve to provide focus correction of the charged particle beam in real time as exposure proceeds. The frequency with which the field can be varied generally is in the kilohertz range.

A liner tube must be configured using a non-magnetic material with low electrical conductivity to allow an AC magnetic field, generated outside the liner tube, to act on a charged particle beam inside the liner tube. Use of materials having low electrical conductivity reduces delays in the control of the charged particle beam that otherwise would exist due to eddy currents generated on the liner tube in response to changes in the magnetic field. For this reason, there are numerous instances of non-conductors, such as ceramics, being used for liner tubes.

Whenever a charged particle beam propagates inside a liner tube, a substantial number of charged particles of the beam collide with the inside-diameter wall of the liner tube. If the liner tube is a non-conductor, then collision of charged particles with a location on the inside-diameter wall causes an accumulation of electrical charge at the location. The accumulated electrical charge, in turn, produces an electrical field that adversely affects the charged particle beam by causing an undesired beam displacement or shift.

If the liner tube contains any deposits of organic material on its inside-diameter surface, collision of the charged particle beam with the organic material generates carbon contamination of the inside-diameter surface. The carbon contamination is susceptible to irregular charge-up and/or discharge. The resulting unwanted varying electrical field can disrupt the beam. Based on the above, it is preferable that contact of the non-conducting, inside-diameter surface of a liner tube with a charged particle beam be avoided. To prevent such contact, certain CPB microlithography apparatus employ a “beam tube” located usually coaxially inside the liner tube. The beam tube is made of a material having a moderate amount of electrical conductivity so as to reduce accumulations of electrical charge or contamination. As used in this context, a “moderate amount of electrical conductivity” means having sufficient electrical conductivity to prevent charge accumulation and having sufficiently low electrical conductivity to prevent significant control delays due to eddy currents in the beam tube. The numerical value of this moderate amount of electrical conductivity can vary, depending upon the desired response speed of the CPB control system in the apparatus, upon the manner in which eddy currents flow in the beam tube, and upon the desired shape of the applied magnetic field. Representative beam-tube materials having desirable electrical conductivities include silicon carbide, graphite, and titanium alloys.

Despite advances as summarized above, relentless demand for increasingly higher performance from microlithography tools has resulted in ongoing demand for apparatus exhibiting increasingly greater speed of CPB deflection, such as for dynamic focus correction. A major impediment to achieving this goal is the above-mentioned response delay due to eddy currents being generated in the beam tube from externally applied magnetic and electrical fields.

FIG. 11(A) depicts a beam tube 111 and a toroidal deflector coil 112 situated outside the beam tube 111. Energization of the deflector coil 112 using AC power causes the deflector coil 112 to produce a magnetic field indicated by the lines of magnetic force 113. As an AC-generated magnetic field, the magnetic field indicated by the lines 113 of magnetic flux is always changing. As indicated by the lines 113, the magnetic field generated by the toroidal coil 112 penetrates the wall of the beam tube 111 to act on the charged particle beam propagating axially through the beam tube 111. As the magnetic field penetrates the wall of the beam tube 111, an eddy current 114 is generated in the wall of the beam tube 111. This eddy current 114 tends to dampen the desired changes to the magnetic field and causes control delays. In FIG. 11(B), the hatched region 115 is an “eddy-current zone” located inside a loop of the eddy current 114. Reducing the electrical conductivity of the beam tube 111 tends to disfavor generation of eddy currents 114, thereby reducing control delays imposed by the eddy currents 114.

Unfortunately, reducing the electrical conductivity of the beam tube tends to increase local charge accumulation inside the beam tube 111, thereby making it impossible for the beam tube to function properly. In addition, the selection of suitable materials from which to make the beam tube is extremely limited because not only must the material have a sufficiently low electrical conductivity but also the material must be compatible with the apparatus.

Another conventional method for reducing control-signal delays through the beam tube is to make the beam tube thinner. By making the beam tube thinner, the cross-sectional area of the beam tube in which eddy currents flow is reduced correspondingly. The resulting increase in electrical resistance makes it difficult for eddy currents to flow. However, there are practical limits on how thin the beam tube can be.

In many CPB microlithography apparatus, the CPB optical system exhibits field curvature, requiring that focus be corrected whenever the beam is deflected. Correcting focus in this manner requires use of a dynamic-focusing coil that is AC-energized. Dynamic-focusing coils tend to experience signal-delay problems if eddy currents are present.

FIG. 12(A) depicts a beam tube 121 and a circumferentially arranged dynamic-focusing coil 126. When energized (usually by AC power), the dynamic-focusing coil 126 generates a magnetic field indicated by lines 123 of magnetic force. As the magnetic field interacts with the beam tube 121, an eddy current 124 is generated inside the wall of the beam tube 121. The lines 123 of magnetic flux pass through the wall of the beam tube 121 to act on the charged particle beam propagating within the beam tube 121. Meanwhile, the eddy current 124 inhibits changes being made to the magnetic field. In FIG. 12(B), the hatched portion 125 is an eddy-current zone situated within the loop 124 of eddy current. As discussed above, the material used to make the beam tube 121 can be selected, and the beam tube 121 can be made thin, so as to minimize signal delays due to the eddy current 124. However, both these approaches have problems as noted above.

### SUMMARY OF THE INVENTION

In view of the shortcomings of the prior art as summarized above, an object of the invention is, inter alia, to provide a charged-particle-beam (CPB) microlithography apparatus exhibiting an increased CPB control response than conventional apparatus, as achieved by reducing eddy currents in the beam tube without creating conditions favoring charge accumulation within or on the beam tube.

To such end, and according to a first aspect of the invention, CPB optical systems are provided. An embodiment of such a system comprises a liner tube (defining an interior space) and a beam tube. The beam tube is situated, within the interior space defined by the liner tube, relative to a coil that tends to generate; whenever the coil is electrically energized, an eddy-current loop in the beam tube. The eddy-current loop defines an eddy-current zone. The beam tube defines at least one slit situated in the eddy-current zone and configured so as to reduce the eddy current.

The at least one slit can extend either part way or entirely through the thickness dimension of the beam tube. I.e., it is not necessary that the slit extend entirely through the thickness dimension. A slit extending part way through the thickness dimension also is effective in reducing the eddy current.

In this configuration, the eddy current in the eddy-current loop is disrupted by the slit(s) provided in the beam tube. As a result, at least a portion of the eddy current is blocked. Blocking the eddy current in this manner tends to divide and thus reduce the overall size of the eddy-current zone. As a result of reducing the size of the eddy-current zone, the response rate at which beam manipulations can be made by deflectors and the like is increased.

The at least one slit can extend parallel to an axis of the beam tube in the eddy-current zone (such a slit is termed a “longitudinal” slit). Especially if multiple eddy-current zones are present in a region of the beam tube, the beam tube desirably includes multiple slits (at least one longitudinal slit per eddy-current zone). Each such longitudinal slit can extend through the wall-thickness dimension at an angle relative to a radius of the beam tube at the respective slit, so as to prevent scatt red charged particles from propagating from inside the beam tube through the respective slit to outside the beam tube. A single longitudinal slit located in an eddy-currnt zone divides the eddy-current zone into two eddy-current zones having a total area less than the area of the undivided eddy-current zone. By dividing the eddy-current zone, the overall eddy current in the beam tube is reduced.

In an alternative configuration, in an eddy-current zone, at least a first slit (longitudinal slit) extends parallel to an axis of the beam tube, and at least a second slit (lateral slit) extends perpendicularly to the first slit. At least one slit can extend through the wall-thickness dimension at an angle relative to a radius of the beam tube at the respective slit, so as to prevent scattered charged particles from propagating from inside the beam tube through the slits to the liner tube.

In yet another alternative configuration, the at least one slit extends at least part way circumferentially around the beam tube. The slit can extend through the wall-thickness dimension at an angle relative to a radius of the beam tube at the slit, so as to prevent charged particles from propagating from inside the beam tube through the slits to the liner tube.

According to another aspect of the invention, beam tubes are provided (in the context of a CPB optical system). Such a beam tube is situated relative to a coil that tends to generate, whenever the coil is electrically energized, an eddy current in an eddy-current zone in the beam tube. The beam tube defines at least one slit situated in the eddy-current zone and configured so as to reduce the eddy current, as summarized above.

According to yet another aspect of the invention, methods are provided for reducing the eddy current in an eddy-current zone of a beam tube of a CPB optical system. In an embodiment of the method, in the eddy-current zone of the beam tube, at least one slit is defined. The slit(s) is situated so as to split the eddy-current zone and thus disrupt (and reduce) the eddy current. The slit(s) can be defined so as to extend through the wall-thickness dimension of the beam tube. The slit(s) can be defined so as to extend parallel to an axis of the beam tube. The slit(s) can be defined so as to extend through the wall-thickness dimension at an angle relative to a radius of the beam tube at the respective slit, so as to prevent scattered charged particles from propagating from inside the beam tube through the respective slit. Other slit configurations can be as summarized above.

The foregoing and additional features and advantages of the invention will be more readily apparent from the following detailed description, which proceeds with reference to the accompanying drawings.

### BRIEF DESCRIPTION OF THE DRAWINGS

FIG. 1(A) is an oblique elevational view of a beam tube and toroidal deflector coil according to a first representative embodiment of the invention.

FIG. 1(B) is an elevational view showing eddy-current zones of the beam tube of FIG. 1(A).

FIG. 2(A) is an oblique elevational view of a beam tube and toroidal deflector coil according to a second representative embodiment of the invention.

FIG. 2(B) is an elevational view showing eddy-current zones of the beam tube of FIG. 2(A).

FIG. 3(A) is an oblique elevational view of a beam tube and dynamic-focus coil according to a third representative embodiment of the invention.

FIG. 3(B) is a transverse section of the beam tube of FIG. 3(A), showing eddy-current zones.

FIG. 4(A) is a transverse section of a beam tube according to a fourth representative embodiment of the invention.

FIG. 4(B) is an oblique view showing how the slits shown in FIG. 4(A) are produced by discharge machining.

FIG. 5(A) is a tranverse section of a beam tube located within a liner tube, wherein the beam tube includes radially oriented slits and showing the trajectories of charged particles through the slits to the liner tube.

FIG. 5(B) is a transverse section of a beam tube located within a liner tube, wherein the beam tube includes slits as shown in FIG. 4(A) and showing how the slits prevent charged particles from propagating radially through the slits to the liner tube.

FIG. 6(A) is an elevational oblique view of a beam tube according to a fifth representative embodiment of the invention.

FIG. 6(B) is an elevational section of the beam tube of FIG. 6(A) situated inside a liner tube, and showing trajectories of charged particles relative to slit; in the beam tube.

FIG. 7(A) is an elevational oblique view of a beam tube according to a sixth representative embodiment of the invention.

FIG. 7(B) is an elevational section of the beam tube of FIG. 7(A) situated inside a liner tube, and showing trajectories of charged particles relative to slits in the beam tube.

FIG. 8 is a transverse section of a beam tube according to the seventh representative embodiment, including slits that do not penetrate the wall of the beam tube.

FIG. 9 is a flow chart of representative steps in a method for manufacturing a microelectronic device.

FIG. 10 is a flow chart of the microlithography step in the process of FIG. 9.

FIG. 11(A) is an oblique elevational view of a conventional beam tube and external toroidal deflector, and showing the magnetic field generated by the toroidal coil and the eddy current generated in the wall of the beam tube.

FIG. 11(B) is an elevational view of the beam tube of FIG. 11(A), depicting the eddy current.

FIG. 12(A) is an oblique elevational view of a conventional beam tube and an external dynamic-focus coil, and showing the magnetic field generated by the dynamic-focus coil and the eddy current generated in the wall of the beam tube.

FIG. 12(B) is an elevational view of the beam tube of FIG. 12(A), depicting the eddy current.

### DETAILED DESCRIPTION

The invention is described below in the context of multiple representative embodiments. It will be understood that the invention is not limited to these embodiments.

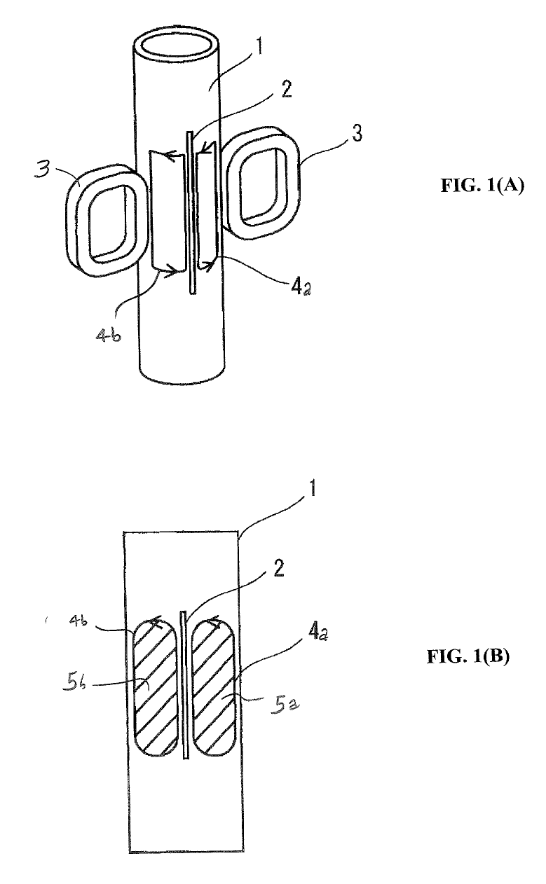

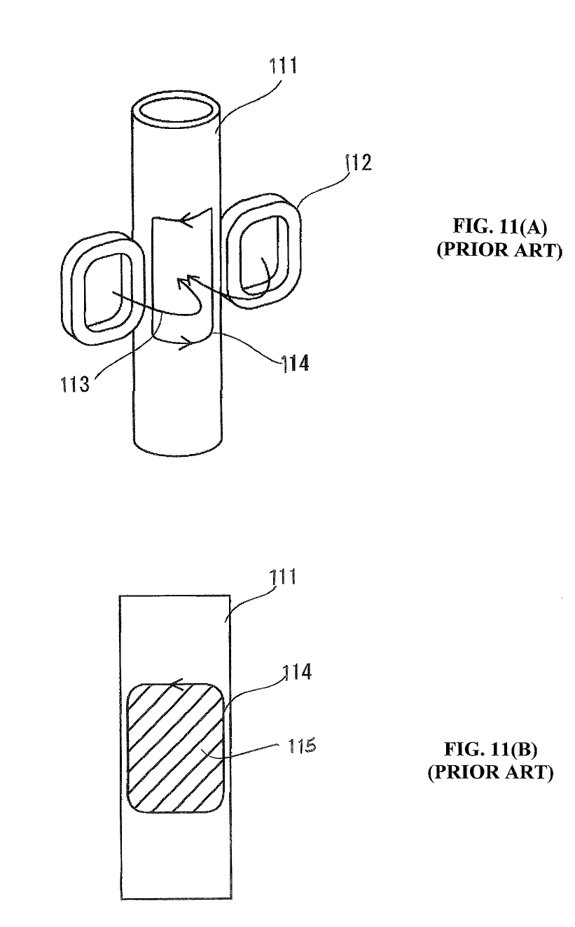

A first representative embodiment is shown in FIGS. 1(A)-1(B). The depicted beam tube 1 is flanked by a toroidal deflector coil 3 in a manner similar to that shown in FIG. 11(A). The beam tube 1 and toroidal deflector coil 3 generally are part of a charged-particle-beam (CPB) optical system. The beam tube 1 defines a longitudinal slit 2 extending parallel to an axis of the beam tube 1 and through the wall of the beam tube 1. The eddy-current loop that would have been formed in the beam tube 1 (lacking slits) by the toroidal coil 3 is divided by the slit 2 into two eddy-current loops 4a, 4b. Although not visible in FIG. 1(A), the beam tube 1 in this embodiment actually defines two symmetrical, diametrically opposed slits 2.

Each slit 2 can be produced by conventional machining of the beam tube 1 (e.g., using a cutting tool or by electrical-discharge machining). As a result of dividing the respective eddy-current loop, each slit 2 defines two eddy-current zones 5a, 5b, as shown in FIG. 1(B). The total area of the eddy-current zones 5a, 5b of FIG. 1(B) is approximately 2/5 the area of the eddy-current zone 115 of a conventional beam tube (lacking slits) as shown in FIG. 11(B). By reducing the total area of the eddy-current zones, overall eddy current in the beam tube 1 is reduced correspondingly. With this first representative embodiment, the 2/5 reduction in eddy current yields a reduction of beam-control delays (due to eddy currents) to approximately 2/5 of what is experienced with the conventional device shown in FIG. 11(B).

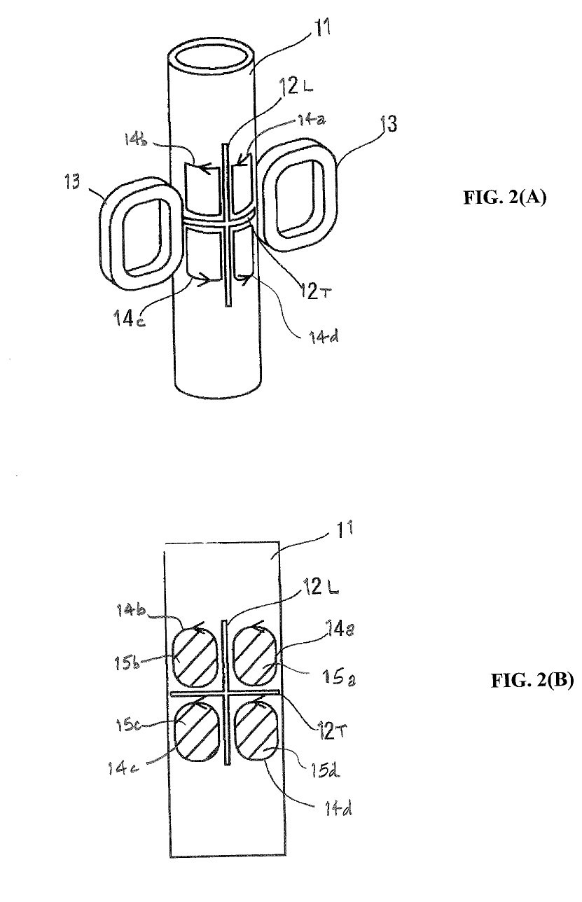

A second representative embodiment is shown in FIGS. 2(A)-2(B). The depicted beam tube 11 is flanked by a toroidal deflector coil 13 in a manner similar to that shown in FIG. 11(A). The beam tube 11 defines a longitudinal slit 12L (extending parallel to an axis of the beam tube 11) and a transverse slit 12T extending through the wall of the beam tube 11. The toroidal coil 13 produces an eddy-current loop in the wall of the beam tube 11, but the slits 12L, 12T divide the eddy-current loop into four eddy-current loops 14a-14d. Although not visible in FIG. 2(A), the beam tube 11 actually defines two symmetrical, diametrically opposed sets of slits 12L, 12T.

Each slit 12L, 12T can be produced by conventional machining of the beam tube 11 (e.g., using a cutting tool or by electrical-discharge machining). As a result of dividing the respective eddy-current loop, each pair of slits 12L, 12T defines four eddy-current zones 15a, 15b, 15c, 15d as shown in FIG. 2(B). Each eddy-current zone 15a-15d of FIG. 2(B) has a respective area that is approximately 1/6 the area of the eddy-current zone 115 of a conventional beam tube (lacking slits) as shown in FIG. 11(B). By reducing the total area of the eddy-current zones, overall eddy current in the beam tube is reduced correspondingly. With this second representative embodiment, the 1/6 reduction in eddy current yields a reduction in beam-control delays (due to eddy currents) to approximately 1/6 of what is experienced with the conventional device shown in FIG. 11(B).

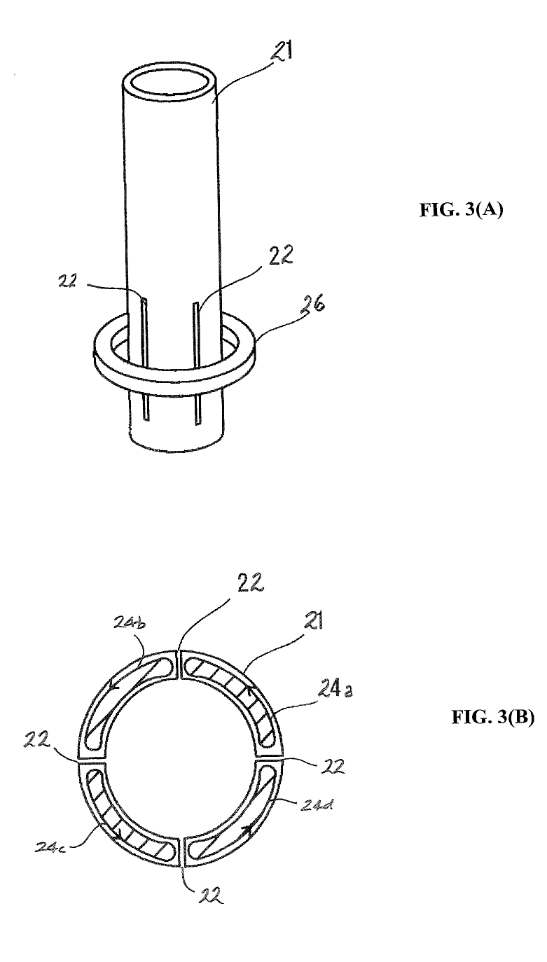

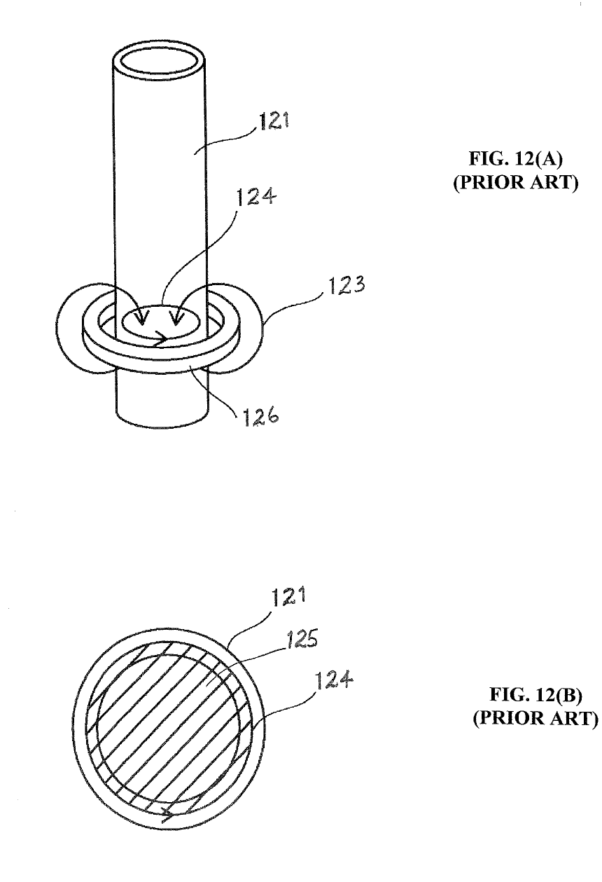

A third representative embodiment is shown in FIGS. 3(A)-3(B). The depicted beam tube 21 is surrounded by a dynamic-focus coil 26 in a manner similar to that shown in FIG. 12(A). The beam tube 21 defines four longitudinal slits 22 extending through the wall of the beam tube 21. The dynamic-focus coil 26 produces an eddy-current loop in the wall of the beam tube 21, but the slits 22 divide the eddy-current loop into four eddy-current loops 24a-24d. The four slits 22 are symmetrically arranged equi-angularly (at every 90°) around the beam tube 21 (FIG. 3(B)).

Each slit 22 can be produced by conventional machining of the beam tube 21 (e.g., using a cutting tool or by electrical-discharge machining). As a result of dividing the eddy-current loop, the slits 22 define four eddy-current zones 25a, 25b, 25c, 25d, as shown in FIG. 3(B). As can be understood by comparing FIG. 3(B) with FIG. 12(B), the total area of the eddy-current zones 25a-25d in FIG. 3(B) is substantially reduced compared to the area 125. As a result, the signal delay of the dynamic-focus coil 26 is greatly improved in this embodiment compared to the conventional device of FIG. 12(B).

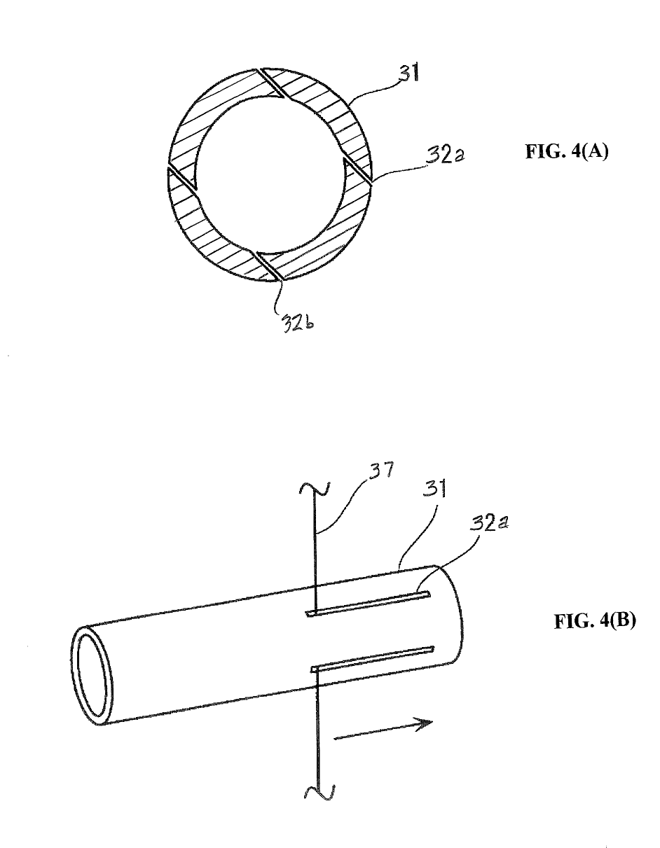

A fourth representative embodiment is shown in FIGS. 4(A)-4(B). The depicted beam tube 31 defines two sets of longitudinal slits 32a, 32b. This embodiment is similar to the third representative embodiment discussed above except for the orientation of the slits 32a, 32b.

The slits 32a, 32b desirably are formed by electrical discharge machining using a wire 37 (FIG. 4(B)) held stationary while moving the beam tube 31 (arrow). Comparing FIG. 4(A) with FIG. 3(A), it can be seen that, in this fourth representative embodiment, the sets 32a, 32b of slits have a different orientation than the slits 22 of FIG. 3(A). I.e., rather than being radially oriented relative to the wall of the beam tube 31, the slits 32a, 32b are angled relative to the radius of the beam tube 31.

The slits 32a, 32b can be produced easily by first drilling holes (at the same orientation as the intended slits and at ends of the intended slits), inserting the wire, and then energizing the wire 37 to perform electrical-discharge machining of the slits 32a, 32b while moving the beam tube (arrow in FIG. 4(B)). The electrical-disharge machining is performed while keeping the wire 37 perpendicular to the axial direction of the beam tube 1. Alternatively to electrical discharge machining, the slits 32a, 32b can be produced by wire cutting or other conventional machining technique.

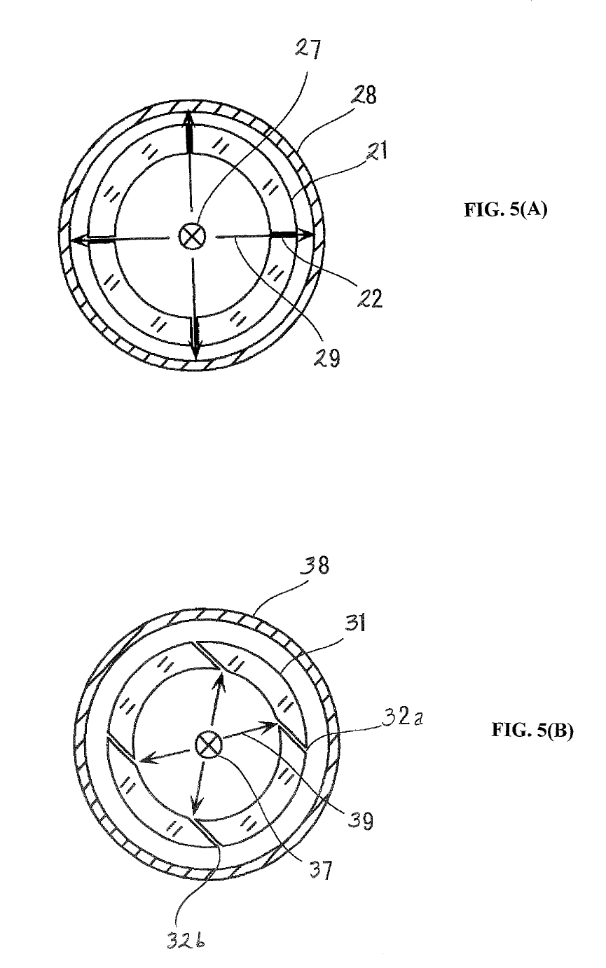

FIG. 5(A) shows the effect when the slits 22 (third representative embodiment) are angled radially (i.e., in the direction of the wall thickness of the beam tube 21). In this figure, item 27 is a charged particle beam (e.g., electron beam), item 28 is a liner tube, and item 29 represents exemplary trajectories of scattered charged particles. The charged particle beam 27 propagates generally along the axis of the beam tube 21. As the beam propagates, charged particles in the beam strike each other and are scattered. Some of the charged particles exhibiting a large scattering angle propagate (trajectories 29) to the slits 22. With slits 22 that are oriented radially through the thickness dimension of the beam tube 21, as shown in FIG. 5(A), scattered charged particles pass through the slits 22. These charged particles can cause accumulation of charges and contaminants on the inside walls of the liner tube 28.

In contrast, if the slits 32a, 32b are angled to the radius (FIG. 5(B)), then scattered charged particles propagating radially (arrows 39) will not pass easily through the slits 32a, 32b. As a result, accumulation of charges and contaminants (e.g., carbon) on the inside wall of the liner tube 38 is prevented. Incidentally, this type of oblique machining of the slits can be applied readily to the longitudinal slits shown in FIGS. 1(A) and 2(A).

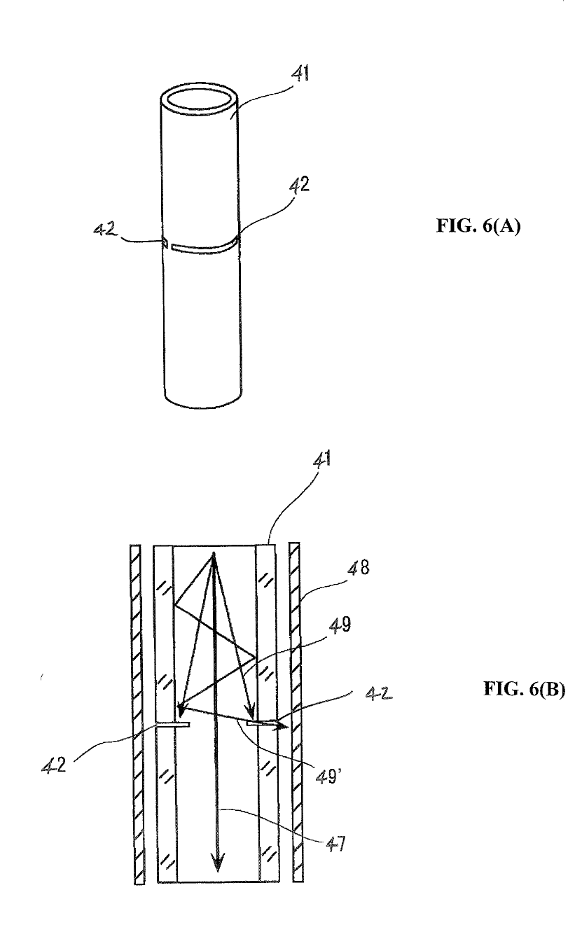

A fifth representative embodiment of a beam tube 41 is shown in FIGS. 6(A)-6(B). The beam tube 41 can be used in association with a toroidal deflector coil as shown in FIG. 11(A), for example. The beam tube 41 defines two half-diameter slits 42 extending circumferentially almost half-way around the beam tube 41. The slits 42 have the same functional effect as the slits 2 shown in FIG. 1(A). However, with the slits 42, scattered charged particles can pass through them.

Propagation of scattered charged particles relative to the slits 42 is shown in FIG. 6(B), depicting a charged particle beam 47 propagating axially through the beam tube 41. Charged particles 49 scattered at relatively shallow angles from the beam 47 do not propagate through the slits 42. In contrast, charged particles 49=40 =0 scattered at relatively large angles to the beam 47 experience multiple scattering inside the beam tube 41 and can propagate through the slits 42 to the liner tube 48.

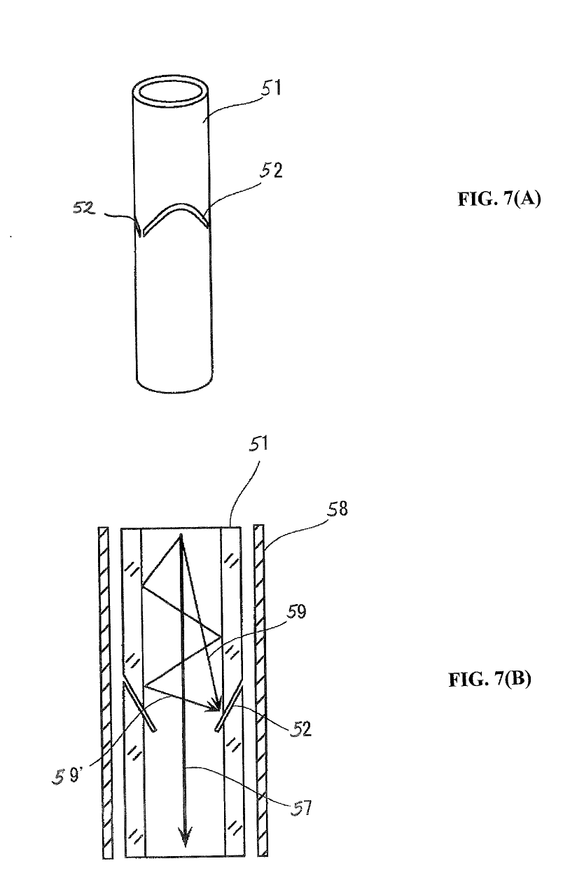

A sixth representative embodiment of a beam tube 51 is shown in FIGS. 7(A)-7(B). This beam tube 51 can be used in association with a toroidal deflector coil as shown in FIG. 11(A), for example, and functions in a manner similar to the beam tube 41 shown in FIG. 6(A). FIG. 7(B) shows that the slits 52 are angled relative to the direction of the wall thickness of the beam tube 51. These slits 52 can be formed by wire cutting, for example, resulting in respective oblique cuts in the beam tube 51.

Passage of scattered charged particles relative to the slits 52 is shown in FIG. 7(B), depicting a charged particle beam 57 propagating axially through the beam tube 51. Charged particles 59 scattered at relatively shallow angles from the beam 57 do not propagate through the slits 52. Also, charged particles 59′ scattered at relatively large angles to the beam 57 do not pass through the slits 52. Thus, the scattered charged particles are prevented from propagating to the liner tube 58, thereby avoiding accumulation of charges and contamination on the inside walls of the liner tube 58.

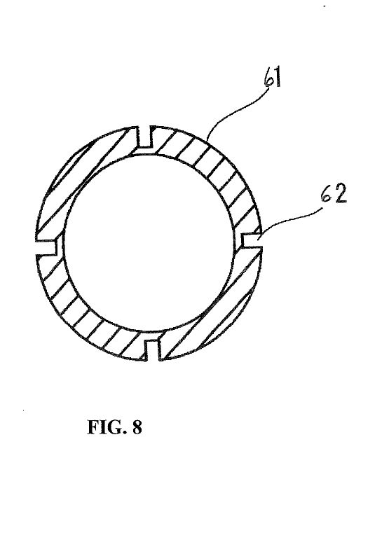

In the first through sixth representative embodiments, the respective slits pass completely through the thickness of the respective beam tube. In the seventh representative embodiment, shown in FIG. 8, eddy-current loops can be divided effectively, as in the other representative embodiments, by using slits 62 that do not pass completely through the thickness dimension of the beam tube 61. A key advantage of this embodiment is that scattered charged particles cannot pass through the slits 62 to the liner tube (not shown).

The representative embodiments described above are exemplary only and can be modified. For example, the number of slits can be selected freely. Further, the slits are not limited to straight-line cuts or criss-crossed configurations. The slits can have any of various shapes and configurations (including bent and zig-zag shapes) appropriate for reducing eddy currents in the beam tubes. The slits also can have any of various shapes and configurations that reflect ease of machining. Also, identical effects can be realized by conjoining portions of liner tubes made of different materials.

In general, any of the representative embodiments described above and within the scope of the invention can be used in a CPB microlithography apparatus that, according to the invention, comprises a CPB optical system including any of said embodiments. The resulting CPB optical systems provide improved beam-control response, thereby allowing microscopic patterns to be transferred with high accuracy. In addition, the CPB optical systems are more resistant, than conventional systems, to accumulation of charges and contaminants on the liner tubes thereof.

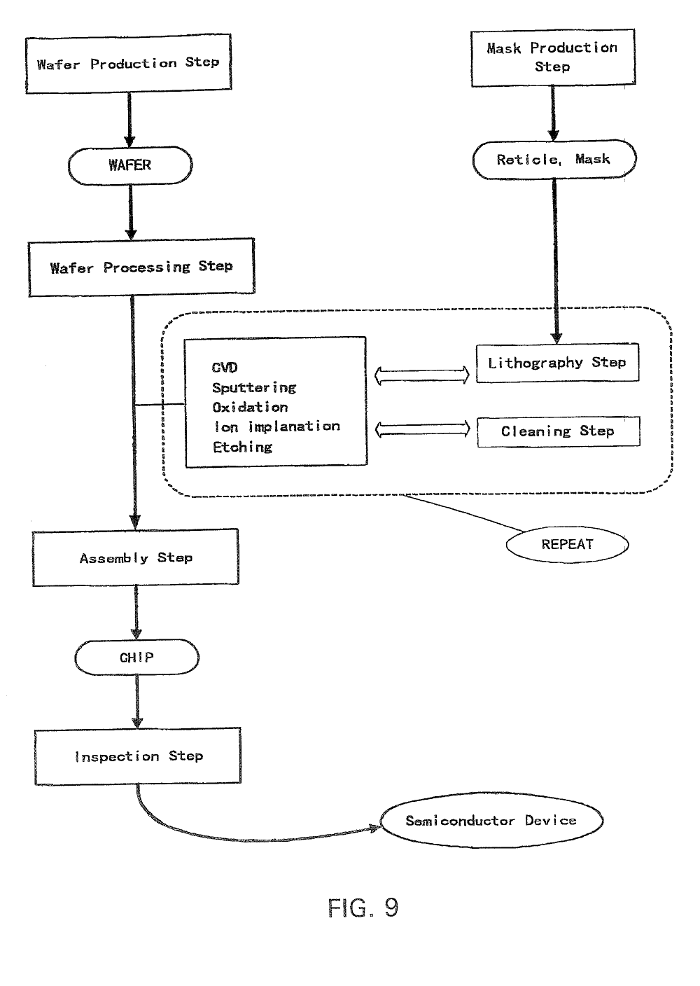

FIG. 9 is a flowchart of an exemplary microelectronic-fabrication method in which apparatus and methods according to the invention can be applied readily The fabrication method generally comprises the main steps of wafer production (wafer manufacturing or preparation), reticle (mask) production or preparation; wafer processing, device (chip) assembly (including dicing of chips and rendering the chips operational), and device (chip) inspection. Each step usually comprises several sub-steps.

Among the main steps, wafer processing is key to achieving the smallest feature sizes (critical dimensions) and best inter-layer registration. In the wafer-processing step, multiple circuit patterns are layered successively atop one another on the wafer, forming multiple chips destined to be memory chips or main processing units (MPUs), for example. The formation of each layer typically involves multiple sub-steps. Usually, many operative microelectronic devices are produced on each wafer.

Typical wafer-processing steps include: (1) thin-film formation (by, e.g., sputtering or CVD) involving formation of a dielectric layer for electrical insulation or a metal layer for connecting wires or electrodes; (2) oxidation step to oxidize the substrate or the thin-film layer previously formed; (3) microlithography to form a resist pattern for selective processing of the thin film or the substrate itself; (4) etching or analogous step (e.g., dry-etching) to etch the thin film or substrate according to the resist pattern; (5) doping as required to implant ions or impurities into the thin film or substrate according to the resist pattern; (6) resist stripping to remove the remaining resist from the wafer; and (7) wafer inspection. Wafer processing is repeated as required (typically many times) to fabricate the desired microelectronic devices on the wafer.

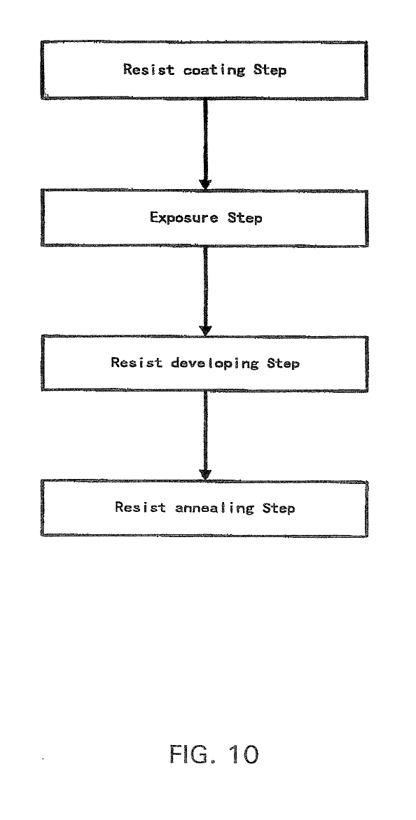

FIG. 10 provides a flowchart of typical steps performed in microlithography, which is a principal step in the wafer-processing step shown in FIG. 7. The microlithography step typically includes: (1) resist-application step, wherein a suitable resist is coated on the wafer substrate (which an include a circuit element formed in a previous wafer-processing step); (2) exposure step, to expose the resist with the desired pattern by microlithography; (3) development step, to develop the exposed resist to produce the imprinted image; and (4) optional resist-annealing step, to enhance the durability of and stabilize the resist pattern. The exposure step can be performed using a CPB microlithography apparatus according to the invention.

The process steps summarized above are all well known and are not described further herein.

Whereas the invention has been described in connection with a representative embodiment, it will be understood that the invention is not limited to that embodiment. On the contrary, the invention is intended to encompass all modifications, alternatives, and equivalents as may be included within the spirit and scope of the invention, as defined by the appended claims.

What is claimed is:

1. A charged-particle-beam (CPB) optical system, comprising:
a liner tube defining an interior space; and
a beam tube situated, within the interior space defined by the liner tube, relative to a coil that tends to generate, whenever the coil is electrically energized, an eddy-current loop in the beam tube, the eddy-current loop defining an eddy-current zone, and the beam tube defining at least one slit situated in the eddy-current zone and configured so as to disrupt the eddy-current loop and thus reduce the eddy current in the beam tube.

2. The system of claim 1, wherein:
the beam tube has a wall-thickness dimension; and
the at least one slit extends through the wall-thickness dimension.

3. The system of claim 1, wherein:
the beam tube has a wall-thickness dimension; and
the at least one slit extends part way through the wall-thickness dimension.

4. The system of claim 1, wherein the at least one slit extends parallel to an axis of the beam tube.

5. The system of claim 4, comprising multiple slits extending parallel to the axis of the beam tube.

6. The system of claim 5, wherein each slit extends, in a direction extending through the wall-thickness dimension, at an angle relative to a radius of the beam tube at the respective slit, so as to prevent scattered charged particles from propagating from inside the beam tube through the slits to the liner tube.

7. The system of claim 1, comprising at least a first slit extending parallel to an axis of the beam tube and a second slit extending perpendicularly to the first slit.

8. The system of claim 1, wherein the at least one slit extends, in a direction extending through the wall-thickness dimension, at an angle relative to a radius of the beam tube at the respective slit, so as to prevent scattered charged particles from propagating from inside the beam tube through the slits to the liner tube.

9. The system of claim 1, wherein the at least one slit extends at least part way circumferentially around the beam tube.

10. The system of claim 9, wherein the at least one slit extends, in a direction extending through the wall-thickness dimension, at an angle relative to a radius of the beam tube at the slit, so as to prevent charged particles from propagating from inside the beam tube through the slits to the liner tube.

11. In a charged-particle-beam (CPB) optical system, a beam tube situated relative to a coil that tends to generate, whenever the coil is electrically energized, an eddy-current loop in the beam tube, the eddy-current loop defining an eddy-current zone, and the beam tube defining at least one slit situated in the eddy-current zone and configured so as to disrupt the eddy-current loop and thus reduce overall eddy current in the beam tube.

12. The system of claim 11, wherein:
the beam tube has a wall-thickness dimension; and
the at least one slit extends through the wall-thickness dimension.

13. The system of claim 11, wherein:
the beam tube has a wall-thickness dimension; and
the at least one slit extends part way through the wall-thickness dimension.

14. The system of claim 11, wherein the at least one slit extends parallel to an axis of the beam tube.

15. The system of claim 14, comprising multiple slits extending parallel to the axis of the beam tube.

16. The system of claim 15, wherein each slit extends, in a direction extending through the wall-thickness dimension, at an angle relative to a radius of the beam tube at the respective slit, so as to prevent scattered charged particles from propagating from inside the beam tube through the slits.

17. The system of claim 11, comprising at least a first slit extending parallel to an axis of the beam tube and a second slit extending perpendicularly to the first slit.

18. The system of claim 1 1, wherein the at least one slit extends, in a direction extending through the wall-thickness dimension, at an angle relative to a radius of the beam tube at the respective slit, so as to prevent scattered charged particles from propagating from inside the beam tube through the slits.

19. The system of claim 11, wherein the at least one slit extends at least part way circumferentially around the beam tube.

20. The system of claim 19, wherein the at least one slit extends, in a direction extending through the wall-thickness dimension, at an angle relative to a radius of the beam tube at the slit, so as to prevent charged particles from propagating from inside the beam tube through the slits.

21. A charged-particle-beam microlithography apparatus, comprising the CPB optical system of claim 1.

22. A charged-particle-beam microlithography apparatus, comprising the CPB optical system of claim 11.

23. In a charged-particle-beam (CPB) method in which a charged particle beam is propagated in an axial direction through a CPB optical system including a beam tube and a coil, wherein the beam tube exhibits an eddy current in an eddy-current zone of the beam tube as caused by energization of the coil, a method for reducing the eddy current in the eddy-current zone, comprising:
in the eddy-current zone of the beam tube, defining at least one slit in the beam tube, the slit being situated so as to split the eddy-current zone and thus disrupt the eddy current.

24. The method of claim 23, wherein:
the beam tube has a wall-thickness dimension; and
the at least one slit is defined so as to extend through the wall-thickness dimension.

25. The system of claim 23, wherein:
the beam tube has a wall-thickness dimension; and
the at least one slit extends part way through the wall-thickness dimension.

26. The method of claim 23, wherein the at least one slit is defined so as to extend parallel to an axis of the beam tube.

27. The method of claim 23, wherein the at least one slit is defined so as to extend, in a direction extending through the wall-thickness dimension, at an angle relative to a radius of the beam tube at the respective slit, so as to prevent scattered charged particles from propagating from inside the beam tube through the slit.

28. The method of claim 23, wherein a first slit is defined so as to extend parallel to an axis of the beam tube, and a second slit is defined so as to extend perpendicularly to the first slit.

29. The method of claim 23, wherein the at least one slit is defined in the beam tube by wire-cutting a wall of the beam tube.

30. The method of claim 23, wherein the at least one slit is defined in the beam tube by electrical-discharge machining.

31. A microelectronic-device fabrication process, comprising:
(a) preparing a substrate;
(b) processing the substrate; and
(c) assembling devices formed on the substrate during steps (a) and (b) h wherein step (b) comprises the steps of (i) applying a resist to the substrate; (ii) exposing the resist; and (iii) developing the resist; and step (ii) comprises providing a CPB microlithography apparatus as recited in claim 22; and using the CPB microlithography apparatus to expose the resist with a pattern.

32. A microelectronic-device fabrication process, comprising:
(a) preparing a substrate;
(b) processing the substrate; and
(c) assembling devices formed on the substrate during steps (a) and (b) wherein step (b) comprises the steps of (i) applying a resist to the substrate; (ii) exposing the resist; and (iii) developing the resist; and step (ii) comprises providing a CPB microlithography apparatus as recited in claim 21; and using the CPB microlithography apparatus to expose the resist with a pattern.
